(19) United States
(12) Patent Application Publication
(10) Pub. No.: US 2010/0312019 A1
(43) Pub. Date: Dec. 9, 2010

(54) Preparation of Methyl Fluoroalkyl Ethers

(76) Inventors: Serguei V. Kovalenko, Martinez, GA (US); Joel Swinson, Evans, GA (US)

Correspondence Address: HOFFMANN & BARON, LLP, 6900 JERICHO TURNPIKE, SYOSSET, NY, 11791, US

(21) Appl. No.: 12/480,390

(22) Filed: Jun. 8, 2009

(51) Int. Cl. C07C 41/01

(52) U.S. Cl. 568/683

(57) ABSTRACT

Methyl fluoroalkyl ether can be produced by the reaction of a fluoroalkyl alcohol with chloromethane. The process involves reacting an alkoxide of a fluoroalkyl alcohol with chloromethane.

### BACKGROUND OF THE INVENTION

Methyl fluoroalkyl ethers are important organic intermediates in the synthesis of other chemical compounds, in particular pharmaceutical or agricultural chemicals. For example, methyl 1,1,1-trifluoroethyl and methyl 1,1,1,3,3,3-hexafluoroisopropyl ethers are used for manufacturing of 2,2,2-trifluoro-1-fluoroethyl-difluoromethyl ether (“desflurane”, International Patent Application No. WO 94/08929), 2,2,2-trifluoro-1-chloroethyl-difluoromethyl ether (“isoflurane”, U.S. Pat. No. 3,535,388 and U.S. Pat. No. 3,535,425) and fluoromethyl 2,2,2-trifluoro-1-(trifluoromethyl)ethyl ether (“sevoflurane” U.S. Pat. No. 3,683,092). Desflurane, isoflurane and sevoflurane are used in modern anesthesiology for human and animal use.

There is a need the development of new, safe, and waste-minimizing processes for the preparation of methyl fluoroalkyl ethers.

### SUMMARY OF THE INVENTION

It is an object of the present invention to provide a very simple industrial process for the preparation methyl fluoroalkyl ethers by reaction of the alkoxides with chloromethane in aqueous solution.

A further advantage of this process is that any unreacted chloromethane may be recovered and recycled for reuse in subsequent reactions, thus reducing process cost and environmental problems associated with the use of dimethyl sulfate. The recovery of chloromethane is relatively simple since it is a low-boiling gas that may be removed from product mixtures by simple distillation.

The invention relates to a process for the preparation of a methyl fluoroalkyl ether. The process comprises reacting an alkoxide of a fluoroalkyl alcohol with chloromethane.

The alkoxide is of the formula (R¹RCH—O⁻)ₙMⁿ⁺, wherein:

R is hydrogen; branched or unbranched, saturated or unsaturated hydrocarbyl having 1-18 carbon atoms in its longest chains; unfused phenyl; fused phenyl having 9-14 carbon atoms; unfused heteroaryl having 5-7 ring atoms; fused heteroaryl having 8-14 ring atoms; unfused, non-aromatic carbocyclic alkyl having 5-8 ring atoms; fused, non-aromatic carbocyclic alkyl having 8-14 ring atoms; unfused, non-aromatic heterocyclic alkyl having 3-8 ring atoms; or fused, non-aromatic heterocyclic alkyl having 8-14 ring atoms;

heteroaryl and heterocyclic alkyl ring atoms are carbon, nitrogen, oxygen, or sulfur;

R¹ is CFXR²;

X is halogen or hydrogen;

R² is halogen, hydrogen, or C₁₋₂ alkyl wherein one or more hydrogen atoms thereof are optionally substituted with an equivalent number of halogen groups;

each hydrocarbyl, phenyl, heteroaryl, carbocyclic alkyl or heterocyclic alkyl, independently, may be unsubstituted or substituted with one or more substituent at any position;

hydrocarbyl substituents are halogen; hydroxyl; hydrocarbyloxy wherein hydrocarbyl is any hydrocarbyl described above; thiol; hydrocarbylthio wherein hydrocarbyl is any hydrocarbyl described above; nitro; carboxyl; NR⁴R⁵; phenyl; unfused heteroaryl having 5-7 ring atoms; or unfused, non-aromatic carbocyclic alkyl or non-aromatic heterocyclic alkyl having 5-8 ring atoms;

phenyl, heteroaryl, carbocyclic alkyl or heterocyclic alkyl substituents are halogen; hydroxyl; nitro; hydrocarbyloxy wherein hydrocarbyl is any hydrocarbyl described above; carboxyl; NR⁴R⁵; C₁₋₆ alkyl; phenyl; unfused heteroaryl having 5-7 ring atoms; or unfused, non-aromatic carbocyclic alkyl or non-aromatic heterocyclic alkyl having 5-8 ring atoms;

halogen groups are fluoro, chloro, bromo, or iodo;

Mⁿ⁺ is an alkali metal ion, an alkaline earth metal ion, or (NR³₄)⁺;

R³ is hydrogen, C₁₋₄ alkyl, or phenyl;

n=1 or 2; and

R⁴ and R⁵ are independently hydrogen, C₁₋₄ alkyl, or phenyl.

In a preferred embodiment, R is hydrogen; branched or unbranched C₁₋₆ alkyl; unfused phenyl; fused phenyl having 9-10 carbon atoms; unfused heteroaryl having 5-6 ring atoms; fused heteroaryl having 9-13 ring atoms; unfused, non-aromatic carbocyclic alkyl having 5-7 ring atoms; fused, non-aromatic carbocyclic alkyl having 9-12 ring atoms; unfused, non-aromatic heterocyclic alkyl having 5-6 ring atoms; or fused, non-aromatic heterocyclic alkyl having 9-13 ring atoms. Most preferably, R is hydrogen, CH₃, or CF₃.

In another preferred embodiment, hydrocarbyl substituents are halogen; hydroxyl; thiol; NR⁴R⁵; phenyl; unfused heteroaryl having 5-6 ring atoms; or unfused, non-aromatic carbocyclic alkyl or non-aromatic heterocyclic alkyl having 5-7 ring atoms. Phenyl, heteroaryl, carbocyclic alkyl or heterocyclic alkyl substituents are halogen; hydroxyl; thiol; nitro; hydrocarbyloxy wherein hydrocarbyl is C₁₋₆ alkyl; carboxyl; NR⁴R⁵; C₁₋₆ alkyl; phenyl; unfused heteroaryl having 5-6 ring atoms; or unfused, non-aromatic carbocyclic alkyl or non-aromatic heterocyclic alkyl having 5-7 ring atoms.

In another preferred embodiment, an aqueous solution of the alkoxide is reacted with chloromethane. The reaction may be carried out in a continuous manner or a batchwise manner.

In one embodiment, the reaction may be carried out at a pressure greater than atmospheric pressure and less than or equal to 600 psi. In another embodiment, the reaction is carried out at atmospheric pressure. In yet another embodiment, the reaction is carried out at a pressure lower than atmospheric pressure and greater than or equal to 0.1 psi.

In a preferred embodiment, M is sodium or potassium. The alkoxide is preferably selected from the group consisting of the sodium and potassium alkoxides of 1,1,1-trifluoroethanol, 1,1,1,3,3,3-hexafluoroisopropanol and 1,1,1-trifluoroisopropanol.

In a preferred embodiment, when the alkoxide is a potassium alkoxide, a 10-80% w/w aqueous solution of potassium alkoxide is reacted with chloromethane. In another preferred embodiment, the amount of chloromethane is from about 70 mol % to about 200 mol % of the potassium alkoxide, and more preferably from about 100 mol % to about 110 mol % of the potassium alkoxide.

In a preferred embodiment, the reaction temperature is from about 20° C. to about 150° C., more preferably from about 70° C. to about 80° C.

### DETAILED DESCRIPTION OF THE INVENTION

According to the invention, a methyl fluoroalkyl ether is produced by the reaction of an alkoxide of a fluoroalkyl alcohol with chloromethane. The alkoxide of a fluoroalkyl alcohol is of the formula (R¹RCH—O⁻)ₙMⁿ⁺ (formula 1).

R is hydrogen; branched or unbranched, saturated or unsaturated hydrocarbyl having 1-18 carbon atoms in its longest chains; unfused phenyl; fused phenyl having 9-14 carbon atoms; unfused heteroaryl having 5-7 ring atoms; fused heteroaryl having 8-14 ring atoms; unfused, non-aromatic carbocyclic alkyl having 5-8 ring atoms; fused, non-aromatic carbocyclic alkyl having 8-14 ring atoms; unfused, non-aromatic heterocyclic alkyl having 3-8 ring atoms; or fused, non-aromatic heterocyclic alkyl having 8-14 ring atoms.

Examples of unbranched, saturated hydrocarbyls having 1-18 carbon atoms include methyl, ethyl, propyl, butyl, hexyl, octyl, decyl, tridecyl, and octadecyl. Examples of branched, saturated hydrocarbyls having 1-18 carbon atoms in their longest chain include 2,2-dimethylpropyl, isobutyl, 2-methylbutyl, tent-butyl, tent-propyl, 3,4,5-trimethyldecyl, 5-propyltridecyl, and 2,4-dimethyloctadecyl.

Examples of unbranched, unsaturated hydrocarbyls having 1-18 carbon atoms include 2-pentenyl, 1-octenyl, 3-octenyl, 4-decenyl, oleyl, linoleyl, and linolenyl. Examples of branched, unsaturated hydrocarbyls having 1-18 carbon atoms include 5-pentyl-3-decenyl, 8-butyl-6-octadecenyl, and 3-ethyl-3-decenyl.

Phenyl is the preferred carbocyclic aryl group.

Unfused heteroaryl and heterocyclic alkyl rings contain 5-7 ring atoms at least one of which is nitrogen, oxygen, or sulfur. Examples of unfused heteroaryls include furanyl, isothiazolyl, pyranyl, pyridinyl, triazolyl, pyridyl, pyrrolyl, thiazolyl, tetrazolyl, pyrazolyl, pyrimidinyl, thiadiazolyl, azepinyl, and diazepinyl.

Unfused, non-aromatic carbocyclic alkyl groups are monocyclic. Examples of unfused, non-aromatic carbocyclic alkyls having 5-8 ring atoms include cyclopentyl, cyclochexyl, cycloheptyl, and cyclooctyl.

Examples of unfused, non-aromatic heterocyclic alkyl groups having 3-8 ring atoms include pyrrolidinyl; tetrahydrofuranyl; 1,2-dioxanyl; 1,3-dioxanyl; 1,4-dioxanyl; piperidinyl; piperazinyl; and morpholinyl.

Each unfused ring discussed above is optionally fused with one or two additional 5-7 member saturated or unsaturated, aromatic or non-aromatic, carbocyclic or heterocyclic rings. Examples of fused rings include naphthyl, carbolinyl, benzimidazolyl, benzofuranyl, benzopyrazolyl, carbazolyl, cinnolinyl, indolyl, benzothiazolyl, benzotriazolyl, bicyclo[4,4,0]decyl, bicyclo[4,3,0]nonyl, 3-aza bicyclo[3,3,0]octyl, and 3-aza bicyclo[4,3,0]nonyl.

Preferably, R in formula 1 is hydrogen; branched or unbranched C₁₋₆ alkyl; unfused phenyl; fused phenyl having 9-10 carbon atoms; unfused heteroaryl having 5-6 ring atoms; fused heteroaryl having 9-10 ring atoms; unfused, non-aromatic carbocyclic alkyl having 5-7 ring atoms; fused, non-aromatic carbocyclic alkyl having 9-10 ring atoms; unfused, non-aromatic heterocyclic alkyl having 5-6 ring atoms; or fused, non-aromatic heterocyclic alkyl having 9-10 ring atoms. Most preferably, R is hydrogen, CH₃, or CF₃.

R¹ is CFXR². X is halogen or hydrogen. Halogen groups include fluoro, chloro, bromo, and iodo. R² is halogen, hydrogen, or C₁₋₂ alkyl wherein one or more hydrogen atoms thereof are optionally substituted with an equivalent number of halogen groups. For example, if R² is C₂ alkyl wherein three hydrogen atoms are substituted with fluoro groups, then R² may be —CH₂CF₃; —CHFCHF₂; or CF₂CFH₂.

Each hydrocarbyl, phenyl, heteroaryl, carbocyclic alkyl or heterocyclic alkyl, independently, may be unsubstituted or substituted with one or more substituent at any position.

Hydrocarbyl substituents are halogen; hydroxyl; hydrocarbyloxy wherein hydrocarbyl is any hydrocarbyl described above; thiol; hydrocarbylthio wherein hydrocarbyl is any hydrocarbyl described above; nitro; carboxyl; NR⁴R⁵; phenyl; unfused heteroaryl having 5-7 ring atoms; or unfused, non-aromatic carbocyclic alkyl or non-aromatic heterocyclic alkyl having 5-8 ring atoms. For example, a hydrocarbyl substituted with a hydroxyl group and a halogen group may be 2-chloro, 5-hydroxyl tridecyl or 2-hydroxyl, 6-bromo octyl.

In a preferred embodiment, hydrocarbyl substituents are halogen; hydroxyl; thiol; NR⁴R⁵; phenyl; unfused heteroaryl having 5-6 ring atoms; or unfused, non-aromatic carbocyclic alkyl or non-aromatic heterocyclic alkyl having 5-7 ring atoms.

Phenyl, heteroaryl, carbocyclic alkyl or heterocyclic alkyl substituents are halogen; hydroxyl; nitro; hydrocarbyloxy wherein hydrocarbyl is any hydrocarbyl described above; carboxyl; NR⁴R⁵; C₁₋₆ alkyl; phenyl; unfused heteroaryl having 5-7 ring atoms; or unfused, non-aromatic carbocyclic alkyl or non-aromatic heterocyclic alkyl having 5-8 ring atoms. R⁴ and R⁵ are independently hydrogen, C₁₋₄ alkyl, or phenyl. For example, a phenyl may be substituted with a hydroxyl group or cyclochexyl may be substituted with a nitro group. An example of a carbocyclic alkyl substituted with halogen and hydrocarbyls is 3-chloro-1,1-dimethylcyclohexane or 2-bromo-3-chloro-1,1-dimethylcyclohexane.

In a preferred embodiment, phenyl, heteroaryl, carbocyclic alkyl or heterocyclic alkyl substituents are halogen; hydroxyl; thiol; nitro; hydrocarbyloxy wherein hydrocarbyl is C₁₋₆ alkyl; carboxyl; NR⁴R⁵; C₁₋₆ alkyl; phenyl; unfused heteroaryl having 5-6 ring atoms; or unfused, non-aromatic carbocyclic alkyl or non-aromatic heterocyclic alkyl having 5-7 ring atoms.

Mⁿ⁺ is an alkali metal ion, an alkaline earth metal ion, or (NR³₄)⁺. R³ is hydrogen, C₁₋₄ alkyl, or phenyl. For example, Mⁿ⁺ describes a cation such as Li⁺, Na⁺, K⁺, Cs⁺, Mg²⁺, Ca²⁺, Sr²⁺, Ba²⁺, or NR′₄⁺. R′ is hydrogen, C₁₋₆ alkyl, aryl, or fluoroalkyl; and n is 1 or 2. Preferably, M is sodium or potassium.

The value of n⁺ in formula 1 depends on M. For alkali metals and quaternary ammonium ions, n⁺=1. For alkaline earth metal ions, n⁺=2. When n⁺=1, n=1. When n⁺=2, n=2.

The alkoxide is preferably selected from the group of sodium or potassium alkoxides of 1,1,1-trifluoroethanol, 1,1,1,3,3,3-hexafluoroisopropanol and 1,1,1-trifluoroisopropanol.

The fluoroalkyl alcohols can be converted to the corresponding alkoxides by methods well known to those skilled in the art. Examples of reagents that will form the alkoxide from the fluoroalkyl alcohol include, but are not limited to, hydroxides, e.g., sodium hydroxide, potassium hydroxide, or a quaternary ammonium hydroxide, carbonates, alkoxides, or hydrides of alkali or alkali earth metals.

The reaction medium in the process according to the invention can be water, organic solvents or water-solvent mixtures. In a preferred embodiment, the process is performed in water. Preferably, the solvent is selected so as to dissolve all of the metal chloride that results from the reaction. The concentration of alkoxides in water is as high as 80 weight % and covers the range 10 to 80 weight %, preferably 50-70 weight %. The increasing of the alkoxide concentration leads to the precipitation of the alkoxide from aqueous solution.

In one embodiment, an aqueous solution of a fluoroalkyl alcohol is mixed with an aqueous solution of metal alkoxide to form a solution of the alkoxide of fluoroalkyl alcohol. However, it is also possible to add a metal alkoxide as a solid to the fluoroalkyl alcohol solution. Preferably, the quantity of metal alkoxide to be added is 1 mol equivalent of the fluorinated alcohols. This reaction is preferably carried out at ambient pressure, and/or in the temperature range from 20° to 60° C., preferably 40° to 50° C.

Chloromethane is added to the reaction vessel containing the solution of the alkoxide of a fluoroalkyl alcohol. The process is not dependent on pressure, so the reaction vessel can be a closed or opened system, and the process may be run at reduced, elevated, or ambient pressure. In a preferred embodiment of the invention, the chloromethane is added to the heated solution of the alkoxide of a fluoroalkyl alcohol in such way that the resulting pressure during the reaction is from 0.1 psi to 600 psi. The reaction may be carried out at a pressure greater than atmospheric pressure and less than or equal to 600 psi; at atmospheric pressure; or at a pressure lower than atmospheric pressure and greater than or equal to 0.1 psi.

Depending on the reaction rate, the temperatures and the apparatus used, chloromethane is added at an appropriate rate within the stated pressure range. For example, the process according to the invention may be carried out in the temperature range from about 20° C. to about 150° C., more preferably from about 50° C. to about 80° C., and most preferably from about 70° C. to about 80° C.

Preferably, the quantity of chloromethane to be added is in range from 70 to 200 mol % of the alkoxide, more preferably from 100 to 110 mol % of the potassium. In another preferred embodiment, a 10-80% w/w aqueous solution of potassium alkoxide is reacted with chloromethane.

The process according to the invention may be run in a continuous manner, or as a batch operation. The preferred manner of operation is continuous for a variety of reasons including improved safety, smaller equipment sizing, space minimization, and increased process efficiency.

In a preferred embodiment of the invention, the alkoxide solution and gaseous chloromethane are concurrently introduced to a reactor. The reagents are allowed to react for an appropriate amount of time, which will vary according to factors such as concentration and reaction temperature, before a crude product stream is removed from the reactor system. Unreacted alkoxide solution and chloromethane may be removed from the crude product and recycled back to the reactor system, using techniques that are well known to those skilled in the art.

The crude product may be isolated and purified by techniques of one of skill in the art of ether synthesis. Such techniques include, but are not limited to, phase separation, washing with water, aqueous acid, and/or aqueous base, solvent extraction, and distillation. The purification process may be run in a continuous manner, or as a batch process. Advantageously, methyl fluoroalkyl ethers are not soluble in water and after completion of the reaction the crude product can be easily separated by simple phase separation. Preferably, a finished product of high purity (typically greater than 99% assay) is obtained by fractional distillation.

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any two, three, or four of the members, e.g., a and c; a, d, and e; b, c, d, and e; etc.

In some cases, the members of a first group of parameters, e.g., a, b, c, d, and e, may be combined with the members of a second group of parameters, e.g., A, B, C, D, and E. Any member of the first group or of a sub-group thereof may be combined with any member of the second group or of a sub-group thereof to form additional groups, i.e., b with C; a and c with B, D, and E, etc.

For example, in the present invention, groups of various parameters are defined (e.g. R, R¹, R², R⁴, and R⁵). Each group contains multiple members. For example, R represents hydrogen; branched or unbranched, saturated or unsaturated hydrocarbyl having 1-18 carbon atoms in its longest chains; unfused phenyl; fused phenyl having 9-14 carbon atoms; unfused heteroaryl having 5-7 ring atoms; fused heteroaryl having 8-14 ring atoms; unfused, non-aromatic carbocyclic alkyl having 5-8 ring atoms; fused, non-aromatic carbocyclic alkyl having 8-14 ring atoms; unfused, non-aromatic heterocyclic alkyl having 3-8 ring atoms; or fused, non-aromatic heterocyclic alkyl having 8-14 ring atoms. Each member may be combined with each other member to form additional sub-groups, e.g., hydrogen, phenyl, and branched or unbranched, saturated or unsaturated hydrocarbyl having 1-18 carbon atoms in its longest chains; or hydrogen, phenyl, and unfused heteroaryl having 5-7 ring atoms.

The instant invention further contemplates embodiments in which each element listed under one group may be combined with each and every element listed under any other group. For example, R is described above. R¹ is CFXR², wherein X is halogen or hydrogen and R² is halogen, hydrogen, or C₁₋₂ alkyl wherein one or more hydrogen atoms thereof are optionally substituted with an equivalent number of halogen groups. Each element of R can be combined with each and every element of R¹. For example, in one embodiment, R may be a propyl and R¹ may be CF₂H. Alternatively, R may be phenyl and R¹ may be CFHCBr₂CBrH₂, etc. Similarly, a third group is Mⁿ⁺, in which the elements are defined as an alkali metal ion, an alkaline earth metal ion, or (NR³₄)⁺, wherein R³ is hydrogen, C₁₋₄ alkyl, or phenyl and n=1 or 2. Each of the above embodiments may be combined with each and every element of M. For example, in the embodiment wherein R is propyl and R¹ is CF₂H, Mⁿ⁺ may be Li⁺, Na⁺, K⁺ (or any other ion within the element of Mⁿ⁺).

With each group, it is specifically contemplated that any one of more members can be excluded. For example, if R² is defined as halogen, hydrogen, or C₁₋₂ alkyl wherein one or more hydrogen atoms thereof are optionally substituted with an equivalent number of halogen groups; it is also contemplated that X is defined as hydrogen or halogen.

The compounds of this invention are limited to those that are chemically feasible and stable. Therefore, a combination of substituents or variables in the compounds described above is permissible only if such a combination results in a stable or chemically feasible compound. A stable compound or chemically feasible compound is one in which the chemical structure is not substantially altered when kept at a temperature of 40° C. or less, in the absence of moisture or other chemically reactive conditions, for at least a week.

A list following the word “comprising” is inclusive or open-ended, i.e., the list may or may not include additional unrecited elements. A list following the words “consisting of” is exclusive or closed ended, i.e., the list excludes any element not specified in the list.

All numbers in the specification are approximate unless indicated otherwise.

### EXAMPLES

The examples which follow illustrate the invention and should not be construed in any way as limiting its scope.

### Example 1

A mixture of 1,1,1,3,3,3-hexafluoroisopropanol (HFIP) (51 g, 0.3 mol) and water (25 ml) is placed in an autoclave (600 ml) and an aqueous solution of potassium hydroxide (45%, 41 g, 0.33 mol) is added at 45°-50° C. for 0.5 hours. Then chloromethane (16 g, 0.32 mol) is added and the reaction mixture is heated at 75° C. for 3 hours with stirring. The reactor is cooled down to room temperature. The organic and aqueous phases are separated. The organic phase is distilled to give pure 1,1,1,3,3,3-hexafluoroisopropyl methyl ether (37 g, 69%) boiling at 50°-51° C.

### Example 2

A mixture of 1,1,1-trifluoroethanol (TFE) (30 g, 0.3 mol) and water (25 ml) is placed in an autoclave (600 ml) and an aqueous solution of potassium hydroxide (45%, 41 g, 0.33 mol) is added at 45°-50° C. for 0.5 hours. Then chloromethane (16 g, 0.32 mol) is added and the reaction mixture is heated at 75° C. for 3 hours with stirring. The reactor is cooled down to room temperature. The organic and aqueous phases are separated. The organic phase is distilled to give pure methyl 1,1,1-trifluoroethyl ether (25 g, 74%) boiling at 30°-31° C.

### Example 3

A mixture of 1,1,1-trifluoroisopropanol (TFIP) (34 g, 0.3 mol) and water (25 ml) is placed in an autoclave (600 ml) and an aqueous solution of potassium hydroxide (45%, 41 g, 0.33 mol) is added at 45°-50° C. for 0.5 hours. Then chloromethane (16 g, 0.32 mol) is added and the reaction mixture is heated at 75° C. for 3 hours with stirring. The reactor is cooled down to room temperature. The organic and aqueous phases are separated. The organic phase is distilled to give pure methyl 1,1,1-trifluoroisopropyl ether (22 g, 58%) boiling at 28°-30° C.

### Example 4

A mixture of HFIP (51 g, 0.3 mol) and water (25 ml) is placed in an autoclave (600 ml) and an aqueous solution of sodium hydroxide (45%, 28 g, 0.33 mol) is added at 45°-50° C. for 0.5 hours. Then chloromethane (16 g, 0.32 mol) is added and the reaction mixture is heated at 75° C. for 3 hours with stirring. The reactor is cooled down to room temperature. The organic and aqueous phases are separated. The organic phase is distilled to give pure 1,1,1,3,3,3-hexafluoroisopropyl methyl ether (31 g, 58%) boiling at 50°-51° C.

1. A process for the preparation of a methyl fluoroalkyl ether, the process comprising reacting an alkoxide of a fluoroalkyl alcohol with chloromethane, wherein the alkoxide is selected from the group consisting of the sodium and potassium alkoxides of 1,1,1-trifluoroethanol, 1,1,1,3,3,3-hexafluoroisopropanol and 1,1,1-trifluoroisopropanol.

2. The process of claim 1, wherein an aqueous solution of the alkoxide is reacted with chloromethane.

3. The process of claim 1, wherein the reaction is carried out in a continuous manner.

4. The process of claim 1, wherein the reaction is carried out in a batchwise manner.

5. The process of claim 1, wherein the reaction is carried out at a pressure greater than atmospheric pressure and less than or equal to 600 psi.

6. The process of claim 1, wherein the reaction is carried out at atmospheric pressure.

7. The process of claim 1, wherein the reaction is carried out at a pressure lower than atmospheric pressure and greater than or equal to 0.1 psi.

8. The process of claim 1, wherein a 10-80% w/w aqueous solution of potassium alkoxide is reacted with chloromethane.

9. The process of claim 8, wherein the amount of chloromethane is from about 70 mol % to about 200 mol % of the potassium alkoxide.

10. The process of claim 9, wherein with the amount of chloromethane is from about 100 mol % to about 110 mol % of the potassium alkoxide.

11. The process of claim 10, wherein the reaction temperature is from about 20° C. to about 150° C.

12. The process of claim 11, wherein the reaction temperature is from about 70° C. to about 80° C.
